(19) United States
(12) Patent Application Publication
(10) Pub. No.: US 2001/0034448 A1
(43) Pub. Date: Oct. 25, 2001

(54) New compounds, their preparation and use

(76) Inventors: Anthony Murray, Hellerup (GB); John Bondo Hansen, Jyderup (DK)

Correspondence Address: Steve T. Zelson, Eqs., Novo Nordisk of North America, Inc., Suite 6400, 405 Lexington Avenue, New York, NY, 10174-6400, US

(21) Appl. No.: 09/835,285

(22) Filed: Apr. 13, 2001

Related U.S. Application Data
(63) Continuation of application No. 09/300,827, filed on Apr. 27, 1999, now abandoned.
(60) Provisional application No. 60/085,884, filed on May 18, 1998.

(30) Foreign Application Priority Data
May 11, 1998 (DK) 0637/98

(51) Int. Cl. C07D 335/06; C07D 311/74; C07D 215/02

(52) U.S. Cl. 546/165; 549/23; 549/396; 564/218

(57) ABSTRACT

The present invention provides novel compounds of formula I

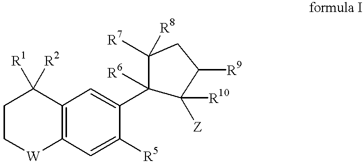

wherein R¹, R², W, Z and R⁵ to R¹⁰ are defined more fully in the description.

The compounds are useful in the treatment of ailments and disorders where a reduction of the blood glucose is beneficial, such as diabetes.

### CROSS-REFERENCE TO RELATED APPLICATIONS

This application claims priority under 35 U.S.C. 119 of U.S. provisional application 60/085,884 filed May 18, 1998 and of Danish application 0637/98 filed May 11, 1998, the contents of which are fully incorporated herein by reference.

### FIELD OF THE INVENTION

The present invention relates to novel compounds, pharmaceutical compositions containing them, methods for preparing the compounds and their use as medicaments. More specifically, compounds of the invention can be utilized in the treatment of conditions mediated by nuclear receptors, in particular the Retinoid X Receptor (RXR) family. The compounds of the invention can also be used in combination with ligands for other nuclear receptors which are known to form dimeric complexes with RXR receptors, for example the Peroxisome Proliferator-Activated Receptor (PPAR) family.

The present compounds reduce blood glucose and triglyceride levels and are accordingly useful for the treatment of ailments and disorders such as diabetes and obesity.

### BACKGROUND OF THE INVENTION

Non-insulin dependent diabetes mellitus (NIDDM, type II diabetes) is a condition characterized by abnormal and ineffective insulin action and secretion. The entry of glucose from the blood into the cells of liver, skeletal muscle and adipose tissue is promoted by insulin action. In the diabetic, tissues dependent on insulin are unable to assimilate glucose normally (insulin resistance), the result being an accumulation of glucose within the blood (hyperglycemia).

Type II diabetes typically afflicts people over 40, and obesity is often a contributing factor. Regulation of diet and exercise can reduce to some extent the problems associated with NIDDM, but commonly insulin therapy or other oral hypoglycemic agents are the treatments of choice.

In addition to the range of insulin formulations, the most widely used hypoglycemic agents to date are sulphonylureas but in respective cases potentially fatal hyperinsulinemia or hypoglycemia can develop, and additional problems involving the cardiovascular, renal, neural and visual systems can also ensue.

More recently, a class of compounds termed thiazolidinediones (e.g. ciglitazone, pioglitazone, englitazone, troglitazone and BRL 49653), have been shown to reduce hyperglycemia by promoting insulin action without additional insulin secretion, and without causing undesirable hypoglycemia, even at elevated doses. Their effect is proposed to be a result of agonism at the PPAR receptor.

Even more recently, it has been reported that RXR agonists such as LGD 1029 and LG 100268 activate RXR/PPAR heterodimers, causing reduction in glucose, insulin and triglyceride levels in ob/ob and db/db mice (Mukherjee et al., Nature 1997, 386, 407-410, Heyman and Mukherjee WO 97/10819). This effect is due to activation at the RXR part of the heterodimer. In turn these RXR/PPAR heterodimers can also be activated by PPAR agonists (e.g. thiazolidinediones) to give a similar effect, and it has been shown that at submaximal levels of either the RXR or PPAR agonist, addition of the complimentary agonist provides an additive and possibly synergistic response, and results in enhanced transcription and subsequently additional lowering of hyperglycemia, hyperinsulinemia and hypertriglyceridemia. It has therefore been proposed that compounds acting as agonists at the RXR receptor can be used as insulin sensitizers for the treatment of type II diabetes and related symptoms, either solely or in combination with PPAR agonists.

### DESCRIPTION OF THE INVENTION

The present invention relates to a compound of formula I

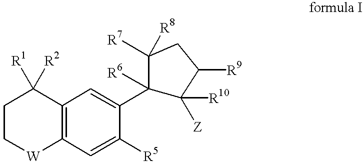

wherein

R¹ and R² are independently hydrogen or C₁₋₆ alkyl;

W is

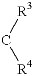

O, N—R³, S, SO or SO₂ wherein R³ and R⁴ are independently hydrogen or C₁₋₆ alkyl;

R⁵ is hydrogen, C₁₋₆ alkyl, halogen, OR¹², SR¹², OCOR¹², NH₂, NHR¹², NR¹²R¹³, NHCOR¹², NR¹²—COR¹³ where R¹² and R¹³ are independently C₁₋₆ alkyl, phenyl or alkyl phenyl;

R⁶ is hydrogen, or taken together with R⁷ forms a double bond, or taken together with R⁷ is methylene to form a cyclopropyl ring;

R⁷ is hydroxy, OR¹³, OCOR¹³ where R¹³ is C₁₋₆ alkyl, phenyl or alkyl phenyl;

R⁸ is hydrogen, C₁₋₆ alkyl or aryl;

R⁹ is hydrogen, or taken together with R¹⁰ forms a double bond, or taken together with R¹⁰ is methylene to form a cyclopropyl ring;

R¹⁰ is hydrogen, hydroxy, OR¹⁴, OCOR¹⁴ where R¹⁴ is C₁₋₆ alkyl, phenyl or alkyl phenyl;

when R⁷, R⁸ and R⁹ are hydrogen, R⁶ and R¹⁰ taken together form a double bond, or taken together are methylene to form a cyclopropyl ring;

when R⁶, R⁹ and R¹⁰ are hydrogen, R⁷ and R⁸ taken together are oxo to form a ketone, or taken together are methylene to form a double bond, or taken together are CH₂CH₂ to form a cyclopropyl ring;

Z is X—Y—R¹¹, wherein X is a valence bond, phenyl or pyridyl, optionally substituted with C₁₋₃ alkyl, halogen, hydroxy, C₁₋₃ alkoxy, C₁₋₃ acyloxy, C₁₋₃ alkyl halide, thiol, C₁₋₃ substituted thiol, Y is C₁₋₆-alkyl, C₂₋₆ alkenyl or C₂₋₆ alkynyl and R¹¹ is CO₂H, tetrazole, PO₃H, SO₃H, CO₂R¹⁶, CONR¹⁷R¹⁸, CH₂OH, CHO, CH₂OR¹⁹, CH(OR²⁰)₂, HC(OR²¹O), COR²², CR²¹(OR²⁰)₂, CR²²(OR²¹O), wherein R¹⁶ is C₁₋₆ alkyl, phenyl or alkyl phenyl; or

Z is ═Y—R¹¹, wherein Y is CR¹⁵, CR¹⁵-C₁₋₆ alkyl, CR¹⁵phenyl, CR¹⁵pyridyl, CR¹⁵C₁₋₃alkylaryl, CR¹⁵-C₂₋₅ alkenyl or CR¹⁵-C₂₋₅ alkynyl, wherein R¹⁵ is H or C₁₋₃ alkyl and R¹¹ is CO₂H, tetrazole, PO₃H, SO₃H, CO₂R¹⁶, CONR¹⁷R¹⁸, CH₂OH, CHO, CH₂OR¹⁹, CH(OR²⁰)₂, HC(OR²¹O), COR²², CR²¹(OR²⁰)₂, CR²²(OR²¹O), wherein R¹⁶ is C₁₋₆ alkyl, phenyl or alkyl phenyl;

R¹⁷ and R¹⁸ are independently hydrogen, C₁₋₆-alkyl, C₅₋₈ cycloalkyl, phenyl or C₁₋₆-alkyl phenyl; R¹⁹ is C₁₋₆-alkyl, phenyl or C₁₋₆-alkyl phenyl; R²⁰ is C₁₋₆ alkyl; R²¹ is C₂₋₄ alkyl; R²² is C₁₋₆ alkyl phenyl or C₃₋₆ cycloalkyl;

or a salt thereof with a pharmaceutically acceptable acid or base, or any optical isomer or mixture of optical isomers, including a racemic mixture, or any tautomeric forms

In the above structural formulas and throughout the present specification, the following terms have the indicated meaning:

The term aryl represents e.g. phenyl, pyridyl, and the like.

The terms “C_{1-n′}-alkyl” wherein n′ can be from 2 through 15, as used herein, represent a branched or straight alkyl group having from one to the specified number of carbon atoms. Typical C₁₋₆-alkyl groups include, but are not limited to, methyl, ethyl, n-propyl, iso-propyl, butyl, iso-butyl, sec-butyl, tert-butyl, pentyl, iso-pentyl, hexyl, iso-hexyl and the like.

The terms “C_{2-n′}-alkenyl” wherein n′ can be from 3 through 15, as used herein, represents an olefinically unsaturated branched or straight group having from 2 to the specified number of carbon atoms and at least one double bond, preferably from one to two double bonds. Examples of such groups include, but are not limited to, vinyl, 1-propenyl, 2-propenyl, allyl, isoproppenyl, 1,3-butadienyl, 1-butenyl, hexenyl, pentenyl, and the like.

The terms “C_{2-n′}-alkynyl” wherein n′ can be from 3 through 15, as used herein, represent an unsaturated branched or straight group having from 2 to the specified number of carbon atoms and at least one triple bond, preferably from one to two triple bonds. Examples of such groups include, but are not limited to, 1-propynyl, 2-propynyl, 1-butynyl, 2-butynyl, 1-pentynyl, 2-pentynyl and the like.

The term cycloalkyl represents e.g. cyclopropyl, cyclobutyl, cyclopentyl and the like.

The term “halogen” means fluorine, chlorine, bromine or iodine.

Certain of the above defined terms may occur more than once in the above formula I, and upon such occurrence each term shall be defined independently of the other.

The compounds of the present invention may have one or more asymmetric centers and it is intended that stereoisomers (optical isomers), as separated, pure or partially purified stereoisomers or racemic mixtures thereof are included in the scope of the invention.

Preferred compounds of the present invention are:

[1-(3,5,5,8,8-Pentamethyl-5,6,7,8-tetrahydro-naphthalen-2-yl)-bicyclo[3.1.0]hex-2-ylidene]-acetic acid

[1-(5,5,8,8-Tetramethyl-5,6,7,8-tetrahydro-naphthalen-2-yl)-bicyclo[3.1.0]hex-2-ylidene]-acetic acid

4-[1-(3,5,5,8,8-Pentamethyl-5,6,7,8-tetrahydro-naphthalen-2-yl)-bicyclo[3.1.0]hex-2-ylidenemethyl]-benzoic acid

4-[1-(5,5,8,8-Tetramethyl-5,6,7,8-tetrahydro-naphthalen-2-yl)-bicyclo[3.1.0]hex-2-ylidenemethyl]-benzoic acid

6-[1-(3,5,5,8,8-Pentamethyl-5,6,7,8-tetrahydro-naphthalen-2-yl)-bicyclo[3.1.0]hex-2-ylidenemethyl]-nicotinic acid

6-[1-(5,5,8,8-Tetramethyl-5,6,7,8-tetrahydro-naphthalen-2-yl)-bicyclo[3.1.0]hex-2-ylidenemethyl]-nicotinic acid

4-[2-(3,5,5,8,8-Pentamethyl-5,6,7,8-tetrahydro-naphthalen-2-yl)-cyclopentylidenemethyl]-benzoic acid

4-[2-(5,5,8,8-Tetramethyl-5,6,7,8,8-tetrahydro-naphthalen-2-yl)-cyclopentylidenemethyl]-benzoic acid

4-[2-(3,5,5,8,8-Pentamethyl-5,6,7,8-tetrahydro-naphthalen-2-yl)-cyclopentylidenemethyl]-nicotinic acid

4-[2-(5,5,8,8-Tetramethyl-5,6,7,8-tetrahydro-naphthalen-2-yl)-cyclopentylidenemethyl]-nicotinic acid

4-[1-(3,5,5,8,8-Pentamethyl-5,6,7,8-tetrahydro-naphthalen-2-yl)-bicyclo[3.1.0]hex-2-en-2-yl]-benzoic acid

4-[1-(5,5,8,8-Tetramethyl-5,6,7,8-tetrahydro-naphthalen-2-yl)-bicyclo[3.1.0]hex-2-en-2-yl]-benzoic acid

6-[1-(3,5,5,8,8-Pentamethyl-5,6,7,8-tetrahydro-naphthalen-2-yl)-bicyclo[3.1.0]hex-2-en-2-yl]-nicotinic acid

6-[1-(5,5,8,8-Tetramethyl-5,6,7,8-tetrahydro-naphthalen-2-yl)-bicyclo[3.1.0]hex-2-en-2-yl]-nicotinic acid

3-[1-(3,5,5,8,8-Pentamethyl-5,6,7,8-tetrahydro-naphthalen-2-yl)-bicyclo[3.1.0]hex-2-en-2-yl]-acrylic acid

3-[1-(5,5,8,8-Tetramethyl-5,6,7,8-tetrahydro-naphthalen-2-yl)-bicyclo[3.1.0]hex-2-en-2-yl]-acrylic acid

[1-(3,5,5,8,8-Pentamethyl-5,6,7,8-tetrahydro-naphthalen-2-yl)-bicyclo[3.1.0]hex-2-en-2-yl]-propynoic acid

[1-(5,5,8,8-Tetramethyl-5,6,7,8-tetrahydro-naphthalen-2-yl)-bicyclo[3.1.0]hex-2-en-2-yl]-propynoic acid

[2-(3,5,5,8,8-Pentamethyl-5,6,7,8-tetrahydro-naphthalen-2-yl)-cyclopent-2-enylidene]-acetic acid

[2-(5,5,8,8-Tetramethyl-5,6,7,8-tetrahydro-naphthalen-2-yl)-cyclopent-2-enylidene]-acetic acid

4-[2-(3,5,5,8,8-Pentamethyl-5,6,7,8-tetrahydro-naphthalen-2-yl)-cyclopent-2-enylidenemethyl]-benzoic acid

4-[2-(5,5,8,8-Tetramethyl-5,6,7,8-tetrahydro-naphthalen-2-yl)-cyclopent-2-enylidenemethyl]-benzoic acid

6-[2-(3,5,5,8,8-Pentamethyl-5,6,7,8-tetrahydro-naphthalen-2-yl)-cyclopent-2-enylidenemethyl]-nicotinic acid

6-[2-(5,5,8,8-Tetramethyl-5,6,7,8-tetrahydro-naphthalen-2-yl)-cyclopent-2-enylidenemethyl]-nicotinic acid

4-[3-Oxo-2-(3,5,5,8,8-pentamethyl-5,6,7,8-tetrahydro-naphthalen-2-yl)-cyclopentyl]-benzoic acid

4-[3-Oxo-2-(5,5,8,8-tetramethyl-5,6,7,8-tetrahydro-naphthalen-2-yl)-cyclopentyl]-benzoic acid

4-[3-Methylene-2-(3,5,5,8,8-pentamethyl-5,6,7,8-tetrahydro-naphthalen-2-yl)-cyclopentyl]-benzoic acid

4-[3-Methylene-2-(5,5,8,8-tetramethyl-5,6,7,8-tetrahydro-naphthalen-2-yl)-cyclopentyl]-benzoic acid

4-[4-(3,5,5,8,8-Pentamethyl-5,6,7,8-tetrahydro-naphthalen-2-yl)-spiro[2.4]hept-5-yl]-benzoic acid

4-[4-(5,5,8,8-Tetramethyl-5,6,7,8-tetrahydro-naphthalen-2-yl)-spiro[2.4]hept-5-yl]-benzoic acid

3-{4-[2-(3,5,5,8,8-Pentamethyl-5,6,7,8-tetrahydro-naphthalen-2-yl)-cyclopent-1-enyl]-phenyl}-but-2-enoic acid

3-{4-[2-(5,5,8,8-Tetramethyl-5,6,7,8-tetrahydro-naphthalen-2-yl)-cyclopent-1-enyl]-phenyl}-but-2-enoic acid

3-{6-[2-(3,5,5,8,8-Pentamethyl-5,6,7,8-tetrahydro-naphthalen-2-yl)-cyclopent-1-enyl]-pyridin-3-yl}-but-2-enoic acid

3-{6-[2-(5,5,8,8-Tetramethyl-5,6,7,8-tetrahydro-naphthalen-2-yl)-cyclopent-1-enyl]-pyridin-3-yl}-but-2-enoic acid

4-[2-(3,5,5,8,8-Pentamethyl-5,6,7,8-tetrahydro-naphthalen-2-yl)-cyclopent-2-enyl]-benzoic acid

4-[2-(5,5,8,8-Tetramethyl-5,6,7,8-tetrahydro-naphthalen-2-yl)-cyclopent-2-enyl]-benzoic acid

6-[2-(3,5,5,8,8-Pentamethyl-5,6,7,8-tetrahydro-naphthalen-2-yl)-cyclopent-2-enyl]-nicotinic acid

6-[2-(5,5,8,8-Tetramethyl-5,6,7,8-tetrahydro-naphthalen-2-yl)-cyclopent-2-enyl]-nicotinic acid

[2-(3,5,5,8,8-Pentamethyl-5,6,7,8-tetrahydro-naphthalen-2-yl)-cyclopent-2-enyl]-propynoic acid

[2-(5,5,8,8-Tetramethyl-5,6,7,8-tetrahydro-naphthalen-2-yl)-cyclopent-2-enyl]-propynoic acid

3-[2-(3,5,5,8,8-Pentamethyl-5,6,7,8-tetrahydro-naphthalen-2-yl)-cyclopent-2-enyl]-acrylic acid

3-[2-(5,5,8,8-Tetramethyl-5,6,7,8-tetrahydro-naphthalen-2-yl)-cyclopent-2-enyl]-acrylic acid

4-[1-Methoxy-2-(3,5,5,8,8-pentamethyl-5,6,7,8-tetrahydro-naphthalen-2-yl)-cyclopentyl]-benzoic acid

4-[1-Methoxy-2-(5,5,8,8-tetramethyl-5,6,7,8-tetrahydro-naphthalen-2-yl)-cyclopentyl]-benzoic acid

6-[1-Methoxy-2-(3,5,5,8,8-pentamethyl-5,6,7,8-tetrahydro-naphthalen-2-yl)-cyclopentyl]-nicotinic acid

6-[1-Methoxy-2-(5,5,8,8-tetramethyl-5,6,7,8-tetrahydro-naphthalen-2-yl)-cyclopentyl]-nicotinic acid

[1-Methoxy-2-(3,5,5,8,8-pentamethyl-5,6,7,8-tetrahydro-naphthalen-2-yl)-cyclopentyl]-propynoic acid

[1-Methoxy-2-(5,5,8,8-tetramethyl-5,6,7,8-tetrahydro-naphthalen-2-yl)-cyclopentyl]-propynoic acid

3-[1-Methoxy-2-(3,5,5,8,8-pentamethyl-5,6,7,8-tetrahydro-naphthalen-2-yl)-cyclopentyl]-acrylic acid

3-[1-Methoxy-2-(5,5,8,8-tetramethyl-5,6,7,8-tetrahydro-naphthalen-2-yl)-cyclopentyl]-acrylic acid

4-[2-Methoxy-1-(3,5,5,8,8-pentamethyl-5,6,7,8-tetrahydro-naphthalen-2-yl)-bicyclo[3.1.0]hex-2-yl]-benzoic acid

4-[2-Methoxy-1-(5,5,8,8-tetramethyl-5,6,7,8-tetrahydro-naphthalen-2-yl)-bicyclo[3.1.0]hex-2-yl]-benzoic acid

6-[2-Methoxy-1-(5,5,8,8-tetramethyl-5,6,7,8-tetrahydro-naphthalen-2-yl)-bicyclo[3.1.0]hex-2-yl]-nicotinic acid

6-[2-Methoxy-1-(3,5,5,8,8-pentamethyl-5,6,7,8-tetrahydro-naphthalen-2-yl)-bicyclo[3.1.0]hex-2-yl]-nicotinic acid

[2-Methoxy-1-(3,5,5,8,8-pentamethyl-5,6,7,8-tetrahydro-naphthalen-2-yl)-bicyclo[3.1.0]hex-2-yl]-propynoic acid

[2-Methoxy-1-(5,5,8,8-tetramethyl-5,6,7,8-tetrahydro-naphthalen-2-yl)-bicyclo[3.1.0]hex-2-yl]-propynoic acid

3-[2-Methoxy-1-(3,5,5,8,8-pentamethyl-5,6,7,8-tetrahydro-naphthalen-2-yl)-bicyclo[3.1.0]hex-2-yl]-acrylic acid

3-[2-Methoxy-1-(5,5,8,8-tetramethyl-5,6,7,8-tetrahydro-naphthalen-2-yl)-bicyclo[3.1.0]hex-2-yl]-acrylic acid

4-[1-Methoxy-2-(3,5,5,8,8-pentamethyl-5,6,7,8-tetrahydro-naphthalen-2-yl)-cyclopent-2-enyl]-benzoic acid

4-[1-Methoxy-2-(5,5,8,8-tetramethyl-5,6,7,8-tetrahydro-naphthalen-2-yl)-cyclopent-2-enyl]-benzoic acid

6-[1-Methoxy-2-(3,5,5,8,8-pentamethyl-5,6,7,8-tetrahydro-naphthalen-2-yl)-cyclopent-2-enyl]-nicotinic acid

6-[1-Methoxy-2-(5,5,8,8-tetramethyl-5,6,7,8-tetrahydro-naphthalen-2-yl)-cyclopent-2-enyl]-nicotinic acid

[1-Methoxy-2-(3,5,5,8,8-pentamethyl-5,6,7,8-tetrahydro-naphthalen-2-yl)-cyclopent-2-enyl]-propynoic acid

[1-Methoxy-2-(5,5,8,8-tetramethyl-5,6,7,8-tetrahydro-naphthalen-2-yl)-cyclopent-2-enyl]-propynoic acid

3-[1-Methoxy-2-(3,5,5,8,8-pentamethyl-5,6,7,8-tetrahydro-naphthalen-2-yl)-cyclopent-2-enyl]-acrylic acid, and

3-[1-Methoxy-2-(5,5,8,8-tetramethyl-5,6,7,8-tetrahydro-naphthalen-2-yl)-cyclopent-2-enyl]-acrylic acid,

or a pharmaceutically acceptable salt thereof.

Pharmaceutically accepted salts of the above invention include pharmaceutically acceptable addition salts, pharmaceutically acceptable metal salts, or optionally alkylated ammonium salts, such as hydrochloric, hydrobromic, hydroiodic, phosphoric, sulphuric, trifluoroacetic, trichloroacetic, oxalic, maleic, pyruvic, malonic, succinic, citric, mandelic, benzoic, cinnamic, methanesulphonic, ethane sulphonic, picric and the like, and include acids related to the pharmaceutically acceptable salts listed (Journal of Pharmaceutical Science 1997, 66, 2) and incorporated herein by reference, or lithium sodium, potassium, magnesium and the like.

The compounds of this invention show a high degree of selectivity towards the RXR receptor family, and in particular have utility for the treatment of symptoms associated with non-insulin dependent diabetes mellitus, either alone or in conjunction with PPAR selective agonists, e.g. thiazolidinediones.

In accordance with the present invention a compound of formula I can be prepared by reacting a compound of formula III (general synthesis described in Beard et al. J. Med Chem 1995, 38, 2820-2829) wherein W and R¹, R² and R⁵ have the meanings as defined for formula. I, with cobalt carbonyl and ethylene in a Pauson Khand reaction (Pauson, Tetrahedron, 1985, 41, 5855)

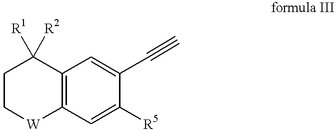

to give a compound of formula IV, wherein W and R¹, R² and R⁵ have the meanings as defined for formula I.

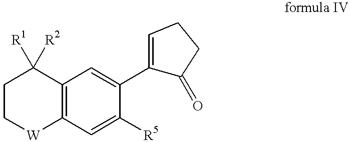

Hydrogenation of a compound of formula IV over a palladium catalyst or cyclopropanation with, for example, dimethyloxosulphonium methylide (Corey et al. J. Am. Chem. Soc. 1963, 1353-1364) to form compounds of formula V, wherein W and R¹, R² and R⁵ to R⁷ have the meanings as defined for formula I.

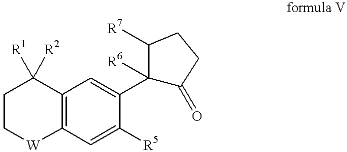

Preparation of, for example, the enol triflate (Ritter Synthesis, 1993, 735) or other group (for example, vinyl halide) capable of participating in a palladium metal mediated cross coupling reaction (Hegedus, in Organometallics in Synthesis, Chapter 5, Wiley 1995 ) of a compound of formula V using triflic anhydride and a suitable base e.g. 2,6-dimethyl pyridine, to form a compound of formula VI where A is OSO₂CF₃ (or alternatively halogen), and wherein W and R¹, R² and R⁵ to R⁷ have the meanings as defined for formula I.

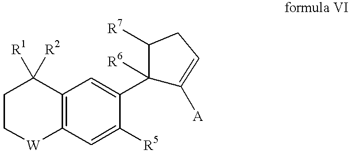

Palladium catalyzed coupling of a compound of formula VI with a suitably metallated (for example, zinc, boron, tin or magnesium) vinyl, aryl, alkynyl or alkyl group according to procedures known in the art to provide a compound of formula VII, wherein W, X, Y, R¹, R² and R⁵ to R⁷ and R¹¹ have the meanings as defined for formula I.

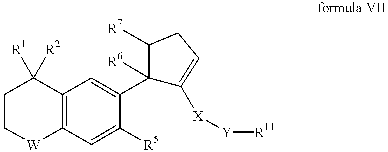

Hydrogenation of a compound of formula VII with hydrogen gas over a palladium catalyst or cyclopropanation of a compound of formula VlI with zinc and diiodomethane according to procedures known in the art to form a compound of formula I.

A compound of formula IV, wherein W and R¹, R² and R⁵ have the meanings as defined for formula I,

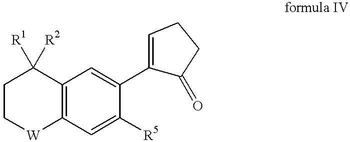

can undergo a Wittig (for example, with an ylide), Horner-Emmons (for example, with a phosphonate) or Reformatsky reaction (for example, with an organozinc reagent) according to procedures known in the art to give a compound of formula VIII

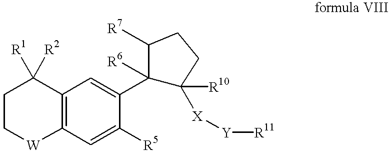

wherein X represents a single bond joining Y to the cycopentane ring and R¹⁰ represents an additional bond to Y, where Y is CR¹⁵-C₀₋₆ alkyl, CR¹⁵phenyl, CR¹⁵pyridyl, CR¹⁵C₁₋₃alkylaryl, CR¹⁵-C₂₋₅ alkenyl having one or two double bonds or CR¹⁵-C₂₋₅ alkynyl having one or two triple bonds where R¹⁵ is H or C₁₋₃ alkyl and W and R¹, R², R⁵ to R⁷ and R¹¹ have the meanings as defined for formula I.

A compound of formula IV, wherein W and R¹, R² and R⁵ have the meanings as defined for formula I,

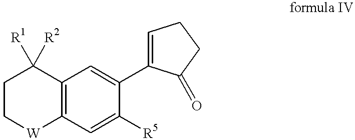

can undergo a 1,4-addition with an alkyl, vinyl or aryl cuprate or zinc reagent (Knochel Tetrahedron 1998, 54, 1471-1490) to give a compound of formula IX

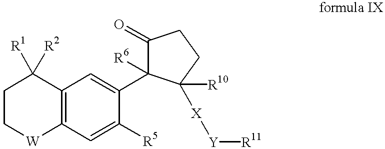

wherein W, X, Y, R¹, R², R⁵, R⁶, R¹⁰ and R¹¹ have the meanings defined for formula I.

Reduction of the cyclopentanone carbonyl of a compound of formula IX using, for example, sodium borohydride, or a Grignard reagent or methylenation and cyclopropanation gives a compound of formula I, where W, X, Y and R¹ through R¹¹ have the meanings defined for formula I.

Reaction of a compound of formula V, wherein W and R¹, R² and R⁵ to R⁷ have the meanings as defined for formula I,

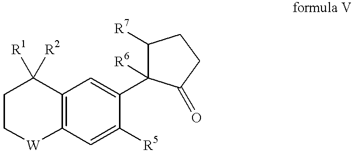

with a Grignard reagent gives a compound of formula X

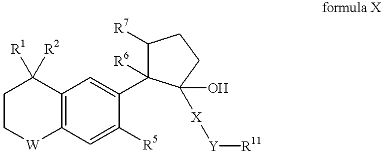

wherein W, X, Y, R¹, R², R⁵ to R⁷ and R¹¹ are as defined for formula I.

Reaction of a compound of formula X with alkyl, aryl halide or acid chloride gives a compound of formula I.

Alcohols can be prepared by reduction of carboxylic acids and derivatives (for example, esters, acid chlorides) with metal hydrides. Aldehydes can be prepared by oxidation of alcohols (for example, with tetrapropyammonium perruthenate or dimethylsulphoxide/oxalyl chloride) or reduction of carboxylic acid esters (for example, with diisobutyl aluminium hydride). Ketones can be prepared by reaction of carboxylic acid derivatives such as N-methyl-N-methoxy amides with Grignard reagents (Weinreb Tet. Lett. 1981, 22, 3815-3819). Ethers can be prepared from alcohols under standard Williamson conditions. Carboxylic acids can be prepared by oxidation of alcohols or aldehydes using mild oxidizing agents (for example, pyridinium dichromate in dimethylformamide).

In cases where a reaction may be inhibited by a reactive functional group contained in the molecule, for example, alcohols, aldehydes, ketones or acids, the corresponding silyl ethers, acetals, ketals or esters can be prepared can be later removed using standard protection/deprotection protocols known in the art. (Kocienski, Protecting Groups, Thieme 1994). In the case of R⁵ being an amino group, protection as an amide by reaction with an activated acyl group is possible; alternatively it is possible to prepare the amino group at a later stage from the corresponding aryl halide by reactions known in the art.

### Molecular Biology Characterization of RXR Activating Compounds

Competitive Binding Assay

The method involves direct interaction between ligand and RXR and was analyzed by displacement of RXR bound [³H] 9-cis RA (retinoic acid) in a competition assay essentially as described (Levin et al. Nature 1992, 355, 359-361 and Heyman et al. Cell 1992, 68, 397-406). Briefly, extracts of infected baculovirus cells expressing recombinant RXRa is used as source of binding activity. The compound of interest is incubated in the presence of [³H] 9-cis RA with RXRa containing extract. Bound probe is separated from unbound through sephadex G50 chromatography. The amount of remaining bound [³H] 9-cis RA was quantitated by scintillation counting.

RXR Transcriptional Activation

The activation potential of a given compound was studied in a transient trans-activation assay, essentially as described (Heyman et al. Cell 1992, 68, 397-406 and Tate et al. Mol. Cel. Biol. 1994, 14, 2323-2330). Expression plasmids encoding RXRa and a DR5 (direct repeat N₅) driven luciferase reporter plasmid was cotransfected into eucaryotic cells. Transfections also contained a plasmid constitutively expressing b-galactosidase (pCMVbgal) and carrier DNA (pGEM). 48 h after transfection cells were washed in PBS and re-fed medium containing ligand or vehicle (DMSO or Ethanol). Following overnight incubation cells were lysed and assayed for luciferase activity. Activation is expressed as the relative amount of luciferase activity (normalized to b-galactosidase activity) in treated versus untreated samples. To determine the specificity of the ligands all were assayed on several nuclear receptors, most notably on RAR. For example, 9-cis retinoic acid (RA) activates both RXR and RAR whereas all-trans RA displays selectivity for RAR, (Heyman et al. Cell 1992, 68, 397-406).

### PHARMACEUTICAL COMPOSITIONS

In another aspect, the present invention includes within its scope pharmaceutical compositions comprising, as an active ingredient, at least one of the compounds of formula I or a pharmaceutically acceptable salt thereof together with a pharmaceutically acceptable carrier or diluent.

Pharmaceutical compositions containing a compound of the present invention may be prepared by conventional techniques, e.g. as described in Remington: The Science and Practice of Pharmacy, 19^{th} Ed., 1995. The compositions may appear in conventional forms such as, for example, capsules, tablets, aerosols, solutions, suspensions or topical applications.

Typical compositions include a compound of formula I or a pharmaceutically acceptable acid addition salt thereof, associated with a pharmaceutically acceptable excipient which may be a carrier or a diluent or be diluted by a carrier, or enclosed within a carrier which can be in Bathe form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier which may be in the form of a ampule, capsule, sachet, paper, or other container. When the carrier serves as a diluent, it may be solid, semi-solid, or liquid material which acts as a vehicle, excipient, or medium for the active compound. The active compound can be adsorbed on a granular solid container, for example, in a sachet. Some examples of suitable carriers are water, salt solutions, alcohols, polyethylene glycols, polyhydroxyethoxylated castor oil, peanut oil, olive oil, gelatin, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavoring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or coloring substances and the like, which do not deleteriously react with the active compounds.

The route of administration may be any route, which effectively transports the active compound to the appropriate or desired site of action, such as oral, nasal, pulmonary, transdermal or parenteral, e.g. rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge . If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound of formula I dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet which may be prepared by conventional tabletting techniques may contain:

| Core: | | |
|---|---|---|
| Active compound (as free compound or salt thereof) | 5 | mg |
| Colloidal silicon dioxide (AEROSIL) | 1.5 | mg |
| Cellulose, microcryst. (AVICEL) | 70 | mg |
| Modified cellulose gum (AC-DI-SOL) | 7.5 | mg |
| Magnesium stearate | Ad. | |
| Coating: | | |
| HPMC approx. | 9 | mg |
| *Mywacett 9-40 T approx. | 0.9 | mg |

*Acylated monoglyceride used as plasticizer for film coating.

The compounds of the invention may be administered to a mammal, especially a human in need of such treatment, prevention, elimination, alleviation or amelioration of diseases related to the regulation of blood sugar.

Such mammals include also animals, both domestic animals, e.g. household pets, and non-domestic animals such as wildlife.

The compounds of the invention are effective over a wide dosage range. For example, in the treatment of adult humans, dosages from about 0.05 to about 100 mg, preferably from about 0.1 to about 100 mg, per day may be used. A most preferable dosage is about 0.1 mg to about 70 mg per day. In choosing a regimen for patients it may frequently be necessary to begin with a dosage of from about 2 to about 70 mg per day and when the condition is under control to reduce the dosage to as low as from about 0.1 to about 10 mg per day. The exact dosage will depend upon the mode of administration, on the therapy desired, form in which administered, the subject to be treated and the body weight of the subject to be treated, and the preference and experience of the physician or veterinarian in charge.

Generally, the compounds of the present invention are dispensed in unit dosage form comprising from about 0.1 to about 100 mg of active ingredient together with a pharmaceutically acceptable carrier per unit dosage.

Usually, dosage forms suitable for oral, nasal, pulmonal or transdermal administration comprise from about 0.001 mg to about 100 mg, preferably from about 0.01 mg to about 50 mg of the compounds of formula I admixed with a pharmaceutically acceptable carrier or diluent.

In a further aspect, the present invention relates to a method of treating and/or preventing type I or type II diabetes.

In a still further aspect, the present invention relates to the use of one or more compounds of formula I or pharmaceutically acceptable salts thereof for the preparation of a medicament for the treatment and/or prevention of type I or type II diabetes.

Any novel feature or combination of features described herein is considered essential to this invention.

### EXAMPLES

The process for preparing compounds of formula I and preparations containing them is further illustrated in the following examples, which however, are not to be construed as limiting.

The structures of the compounds are confirmed by either elemental analysis (MA) nuclear geomagnetic resonance (NMR) or mass spectrometry (MS). NMR shifts (d) are given in parts per million (ppm) and only selected peaks are given. mp is melting point and is given in ° C. Column chromatography was carried out using the technique described by W. C. Still et al., J. Org. Chem. 1978, 43, 2923-2925 on Merck silica gel 60 (Art 9385). Compounds used as starting materials are either known compounds or compounds which can readily be prepared ruby methods known per se.

Abbreviations

TLC: thin layer chromatography

DMSO: dimethylsulfoxide

CDCl₃: deutorated chloroform

DMF: N,N-dimethylformamide

min: minutes

h: hours

### Example 1

### 4-[1-(3,5,5,8,8-Pentamethyl-5,6,7,8-tetrahydro-naphthalen-2-yl)-bicyclo[3.1.0]hex-2-ylidenemethyl]-benzoic Acid

Step 1

To a stirred suspension of cobalt carbonyl (9.1 g, 27 mmol) in petroleum ether (300 mL) under nitrogen was added 6-ethynyl-1,1,4,4,7-pentamethyl-1,2,3,4-tetrahydro-naphthalene (5.5 g, 24 mmol) petroleum ether (25 mL), and the reaction stirred for 3 h at room temperature. The reaction mixture was then filtered through a pad of Celite and concentrated under reduced pressure. The crude cobalt alkyne complex was redissolved in benzene (75 mL) and put under an ethylene atmosphere using a balloon. A solution of trimethylamine-N-oxide in methanol (75 mL) was added over 2.5 h and the reaction was stirred for 16 h. The reaction mixture was then filtered through a pad of Celite and concentrated under reduced pressure, and the residue purified by flash chromatography (eluant 2 hexane:1 ethyl acetate) to give 2-(3,5,5,8,8-pentamethyl-5,6,7,8-tetrahydro-naphthalen-2-yl)-cyclopent-2-enone (2.88 g, 52%).

¹H NMR (CDCl₃, 300 MHz): 1.26 (6H, s), 1.27 (6H, s), 1.65 (4H, s), 2.14 (3H, s), 2.54 (2H, m), 2.62 (2H, m), 7.04 (1H, s), 7.12 (1H, s), 7.58 (1H, m).

¹³C NMR (CDCl₃, 75 MHz): 208.2, 161.2, 147.0, 145.3, 142.5, 133.5, 129.6, 128.7, 127.9, 126.3, 35.5, 35.3, 34.4, 34.3, 32.3, 32.2, 27.2, 20.4.

MS Calcd for C₂₀H₂₆O 282.4, Found 282.3.

Step 2

To a stirred solution of 2-(3,5,5,8,8-pentamethyl-5,6,7,8-tetrahydro-naphthalen-2-yl)-cyclopent-2-enone (0.19 g, 0.67 mmol) in methanol (5 mL) in an ice bath was added cerium chloride heptahydrate (0.32 g, 0.87 mmol) and the whole stirred for 5 min. Sodium borohydride 35 mg, 0.92 mmol) was then added in one portion and the reaction stirred for 15 min. Diethyl ether (15 mL) and a mixture of brine (5 mL) and dilute HCl (1 mL) was added and the organic phase recovered. The aqueous phase was extracted with diethyl ether and the combined organic layers dried over sodium sulphate and concentrated to give a residue, which was purified by flash chromatography (eluant 3 hexane:1 ethyl acetate) to give 2-(3,5,5,8,8-pentamethyl-5,6,7,8-tetrahydro-naphthalen-2-yl)-cyclopent-2-enol (0.19 g, 99%).

¹H NMR (CDCl₃, 300 MHz): 1.26 (12H, s), 1.65 (4H, s), 1.81-1.98 (1 H, m), 2.14 (3H, s), 2.42-2.50 (2H, m), 2.52-2.69 (1H, m), 5.10-5.19 (1H, m), 5.87 (1H, m), 7.10 (1H, s), 7.21 (1H, s).

¹³C NMR (CDCl₃, 75 MHz): 146.6, 144.6, 143.0, 134.0, 133.6, 132.8, 129.4, 127.6, 80.1, 61.3, 36.0, 34.8, 34.7, 34.2, 32.8, 32.7, 32.6, 31.5, 21.5, 15.0.

MS Calcd for C₂₀H₂₈O 284.4, Found 284.3.

Step 3

To a stirred solution of diethylzinc (0.57 mL, 5.5 mmol) dichloroethane (4 mL) in an ice bath was added, dropwise, chloroiodoethane (0.8 mL, 11 mmol) forming a white suspension. After 10 min 2-(3,5,5,8,8-pentamethyl-5,6,7,8-tetrahydro-naphthalen-2-yl)-cyclopent-2-enol (0.4 g, 1.4 mmol) in dichloroethane (1 mL) was added and the reaction stirred at this temperature for 15 min, then heated to 50° C. for 20 min. The reaction mixture was diluted with diethyl ether and saturated ammonium chloride (8 mL) was added. The ether phase was washed with water and dried over sodium sulphate, and concentrated to give a residue, which was purified by flash chromatography (eluant 4 hexane:1 ethyl acetate) to give 1-(3,5,5,8,8-pentamethyl-5,6,7,8-tetrahydro-naphthalen-2-yl)-bicyclo[3.1.0]hexan-2-ol (40 mg, 24%).

¹H NMR (CDCl₃, 300 MHz): 0.73 (1H, q), 1.10 (1H, t), 1.26 (12H, s),1.1.15-1.34 (1H, m), 1.4-1.5 (1H, m), 1.61 (3H, s), 1.79-2.10 (3H, m), 4.5 (1H, t), 7.03 (1H, s), 7.18 (1H, s).

¹³C NMR (CDCl₃, 75 MHz): 143.7, 142.8, 137.6, 135.2, 129.9, 128.6, 126.3, 78.9, 37.5, 35.6, 35.6, 34.3, 32.4, 32.3, 32.2, 29.6, 25.5, 25.3, 19.5, 11.0.

MS Calcd for C₂₁H₃₀O 298.5, Found 298.3.

Step 4

A mixture of 1-(3,5,5,8,8-pentamethyl-5,6,7,8-tetrahydro-naphthalen-2-yl)-bicyclo[3.1.0]hexan-2-ol (40 mg, 0.13 mmol), pyridinium chlorochromate (60 mg, 0.28 mmol) and dichloromathane (3 mL) was stirred for 2 h at room temperature. Removal of solvent under reduced pressure gave a residue, which was purified by flash chromatography (eluant 4 hexane:1 ethyl acetate) to give 1-(3,5,5,8,8-pentamethyl-5,6,7,8-tetrahydro-naphthalen-2-yl)-bicyclo[3.1.0]hexan-2-one (40 mg, 99%) as a white solid mp 152.5-153.5° C.

¹H NMR (CDCl₃, 300 MHz): 1.21 (3H, s), 1.23 (9H, s), 1.59 (1H, t), 1.60 (1H, q), 1.63 (4H, s), 2.02-2.41 (5H, m), 2.29 (3H, s), 7.02 (1H, s), 7.08 (1H, s).

¹³C NMR (CDCl₃, 75 MHz): 213.9, 144.5, 142.4, 135.8, 132.4, 128.8, 128.5, 126.3, 41.7, 35.6, 34.3, 32.8, 32.4, 32.3, 32.2, 29.9, 22.3, 20.3, 19.8.

MS Calcd for C₂₁H₂₈O 296.5, Found 296.2.

Step 5

To a stirred suspension of sodium hydride (60 mg of 60% in mineral oil, 1.4 mmol) in THF (2 mL) under nitrogen was added 4-(diethoxy-phosphorylmethyl)-benzoic acid methyl ester (0.4 g, 1.4 mmol) in THF (1 mL) and the mixture stirred for 20 min. A mixture of 1-(3,5,5,8,8-Pentamethyl-5,6,7,8-tetrahydro-naphthalen-2-yl)-bicyclo[3.1.0]hexan-2-one (70 mg, 0.23 mmol) and 15-crown-5 (0.28 mL, 1.4 mmol) was added and the reaction stirred for 16 h at room temperature. Water was added and the aqueous phase extracted with diethyl ether, the combined organic layers were dried over sodium sulphate, and concentrated to give a residue, which was purified by flash chromatography (eluant 4 hexane:1 ethyl acetate) to give a mixture of 4-[1-(3,5,5,8,8-pentamethyl-5,6,7,8-tetrahydro-naphthalen-2-yl)-bicyclo[3.1.0]hex-2-ylidenemethyl]-benzoic acid ethyl ester, 4-[1-(3,5,5,8,8-pentamethyl-5,6,7,8-tetrahydro-naphthalen-2-yl)-bicyclo[3.1.0]hex-2-ylidenemethyl]-benzoic acid methyl ester and 4-[1-(3,5,5,8,8-pentamethyl-5,6,7,8-tetrahydro-naphthalen-2-yl)-bicyclo[3.1.0]hex-2-en-2-ylmethyl]-benzoic acid ethyl ester (64 mg), which was used directly in the next step.

Step 6

A mixture of 4-[1-(3,5,5,8,8-pentamethyl-5,6,7,8-tetrahydro-naphthalen-2-yl)-bicyclo[3.1.0]hex-2-ylidenemethyl]-benzoic acid ethyl ester, 4-[1-(3,5,5,8,8-Pentamethyl-5,6,7,8-tetrahydro-naphthalen-2-yl)-bicyclo[3.1.0]hex-2-ylidenemethyl]-benzoic acid methyl ester and 4-[1-(3,5,5,8,8-pentamethyl-5,6,7,8-tetrahydro-naphthalen-2-yl)-bicyclo[3.1.0]hex-2-en-2-ylmethyl]-benzoic acid ethyl ester (50 mg, 0.12 mmol) and aqueous potassium hydroxide (0.2 mL of 6M) in methanol (3 mL) was heated at reflux for 2 h. Dilute hydrochloric acid was added and the solvent removed under reduced pressure. The residue was partitioned between dichloromethane (5 mL) and water (5 mL), and the aqueous layer extracted three times with dichloromethane (5 mL). The combined organic layers were dried over sodium sulphate, and concentrated to give a residue, which was purified by flash chromatography (eluant 17 dichloromethane:1 methanol) to give a white solid (42 mg). Recrystallization from hexane/ethyl acetate gave 4-[1-(3,5,5,8,8-pentamethyl-5,6,7,8-tetrahydro-naphthalen-2-yl)-bicyclo[3.1.0]hex-2-ylidenemethyl]-benzoic acid (26 mg) as a white solid mp 274.5-275.2° C.

¹H NMR (CDCl₃, 300 MHz): 1.00-2.48 (remaining H, m) 2.71-2.85 (1H, m), 5.74 (1H, s), 7.10 (1H, s), 7.15-7.3 (3H, m), 7.97 (2H, d).

¹³C NMR (CDCl₃, 75 MHz): 171.8, 153.7, 144.3, 143.4, 142.1, 135.9, 135.4, 129.9, 129.7, 128.2, 128.0, 125.9, 119.6, 40.2, 35.2, 32.0, 31.9, 27.1, 26.9, 26.7, 19.5, 18.2.

MS Calcd for C₂₉H₃₄O₂ 414.6, Found 414.2.

Microanalysis Calcd for C₂₉H₃₄O₂ C 84.02, H 8.33. Found C 84.28, H 8.27.

### Example 2

### [1-(3,5,5,8,8-Pentamethyl-5,6,7,8-tetrahydro-naphthalen-2-yl)-bicyclo[3.1.0]hex-2-ylidene]-acetic Acid Ethyl Ester

To a stirred suspension of sodium hydride (14 mg of 60% in mineral oil, 0.34 mmol) in THF (1 mL) under nitrogen was added triethylphosphonoacetate (0.225 g, 1.0 mmol) in THF (1 mL) and the mixture stirred for 20 min. A mixture of 1-(3,5,5,8,8-pentamethyl-5,6,7,8-tetrahydro-naphthalen-2-yl)-bicyclo[3.1.0]hexan-2-one (50 mg, 0.17 mmol) and 15-crown-5 (0.20 mL, 1.0 mmol) was added and the reaction stirred 72 h at room temperature. The reaction solvent was removed under reduced pressure and water was added. Extraction with diethyl ether, drying over magnesium sulphate and removal of solvent under reduced pressure gave a residue which was purified by flash chromatography to give [1-(3,5,5,8,8-pentamethyl-5,6,7,8-tetrahydro-naphthalen-2-yl)-bicyclo[3.1.0]hex-2-ylidene]-acetic acid ethyl ester (22 mg).

¹H NMR (CDCl₃, 300 MHz): 0.80-2.35 (m, remaining H), 3.4-3.55 (1H, m), 4.01-4.19 (2H, m), 5.2 (1H, s), 7.03 (1H, s), 7.14 (1H, s).

¹³C NMR (CDCl₃, 75 MHz): 167.2, 143.6, 142.2, 135.1, 135.0, 129.6, 128.1, 110.6, 59.5, 35.2, 33.9, 32.0, 31.9, 29.6, 27.6, 26.7, 20.0, 19.5, 14.4.

MS Calcd for C₂₁H₃₄O₂ 366.5, Found 366.2.

What is claimed is:

1. A compound of formula I 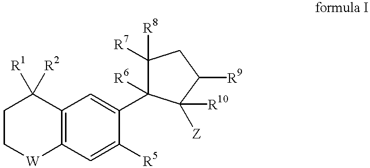
wherein
R¹ and R² are independently hydrogen or C₁₋₆ alkyl;
W is 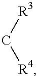
O, N—R³, S, SO or SO₂ wherein R³ and R⁴ are independently hydrogen or C₁₋₆ alkyl;
R⁵ is hydrogen, C₁₋₆ alkyl, halogen, OR¹², SR¹², OCOR¹², NH₂, NHR¹², NR¹²R¹³, NHCOR¹², NR¹²—COR¹³ where R¹² and R¹³are independently C₁₋₆ alkyl, phenyl or alkyl phenyl;
R⁶ is hydrogen, or taken together with R⁷ forms a double bond, or taken together with R⁷ is methylene to form a cyclopropyl ring;
R⁷ is hydroxy, OR¹³, OCOR¹³ where R¹³ is C₁₋₆ alkyl, phenyl or alkyl phenyl;
R⁸ is hydrogen, C₁₋₆ alkyl or aryl;
R⁹ is hydrogen, or taken together with R¹⁰ forms a double bond, or taken together with R¹⁰ is methylene to form a cyclopropyl ring;
R¹⁰ is hydrogen, hydroxy, OR¹⁴, OCOR¹⁴ where R¹⁴ is C₁₋₆ alkyl, phenyl or alkyl phenyl;
when R⁷, R⁸ and R⁹ are hydrogen, R⁶ and R¹⁰ taken together form a double bond, or taken together are methylene to form a cyclopropyl ring;
when R⁶, R⁹ and R¹⁰ are hydrogen, R⁷ and R⁸ taken together are oxo to form a ketone, or taken together are methylene to form a double bond, or taken together are CH₂CH₂ to form a cyclopropyl ring;
Z is X—Y—R¹¹, wherein X is a valence bond, phenyl or pyridyl, optionally substituted with C₁₋₃ alkyl, halogen, hydroxy, C₁₋₃ alkoxy, C₁₋₃ acyloxy, C₁₋₃ alkyl halide, thiol, C₁₋₃ substituted thiol, Y is C₁₋₆-alkyl, C₂₋₆ alkenyl or C₂₋₆ alkynyl and R¹¹ is CO₂H, tetrazole, PO₃H, SO₃H, CO₂R¹⁶, CONR¹⁷R¹⁸, CH₂OH, CHO, CH₂OR¹⁹, CH(OR²⁰)₂, HC(OR²¹O), COR²², CR²¹(OR²⁰)₂, CR²²(OR²¹O), wherein R¹⁶ is C₁₋₆ alkyl, phenyl or alkyl phenyl; or
Z is ═Y—R¹¹, wherein Y is CR¹⁵, CR¹⁵-C₁₋₆ alkyl, CR¹⁵phenyl, CR¹⁵pyridyl, CR¹⁵C₁₋₃alkylaryl, CR¹⁵-C₂₋₅ alkenyl or CR¹⁵-C₂₋₅ alkynyl, wherein R¹⁵ is H or C₁₋₃ alkyl and R¹¹ is CO₂H, tetrazole, PO₃H, SO₃H, CO₂R¹⁶, CONR¹⁷R¹⁸, CH₂OH, CHO, CH₂OR¹⁹, CH(OR²⁰)₂, HC(OR²¹O), COR²², CR²¹(OR²⁰)₂, CR²²(OR²¹O), wherein R¹⁶ is C₁₋₆ alkyl, phenyl or alkyl phenyl;
R¹⁷ and R¹⁸ are independently hydrogen, C₁₋₆-alkyl, C₅₋₈ cycloalkyl, phenyl or C₁₋₆-alkyl phenyl; R¹⁹ is C₁₋₆ -alkyl, phenyl or C₁₋₆-alkyl phenyl; R²⁰ is C₁₋₆ alkyl; R²¹ is C₂₋₄ alkyl; R²² is C₁₋₆ alkyl phenyl or C₃₋₆ cycloalkyl;
or a salt thereof with a pharmaceutically acceptable acid or base, or any optical isomer or mixture of optical isomers, or any tautomeric forms
provided that when R⁵ is C₄₋₆-alkyl and R⁷, R⁸ and R⁹ are hydrogen and R⁶ and R¹⁰ taken together form a double bond, or taken together are methylene to form a cyclopropyl ring, then Z cannot be X—Y—R¹¹, wherein X is a valence bond, Y is C₂₋₆-alkenyl and R¹¹ is CO₂H, CO₂R¹⁶ or COR²², wherein R¹⁶ and R²² is as defined above.

2. A compound according to claim 1 wherein R⁵ is hydrogen or C₁₋₆-alkyl.

3. A compound according to claim 1 wherein W is 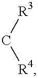
wherein R³and R⁴ are independently C₁₋₆ alkyl.

4. A compound according to claim 1 wherein R⁶ taken together with R⁷ is methylene to form a cyclopropyl ring.

5. A compound according to claim 1 selected from the group consisting of
[1-(3,5,5,8,8-Pentamethyl-5,6,7,8-tetrahydro-naphthalen-2-yl)-bicyclo[3.1.0]hex-2-ylidene]-acetic acid,
[1-(5,5,8,8-Tetramethyl-5,6,7,8-tetrahydro-naphthalen-2-yl)-bicyclo[3.1.0]hex-2-ylidene]-acetic acid,
4-[1-(3,5,5,8,8-Pentamethyl-5,6,7,8-tetrahydro-naphthalen-2-yl)-bicyclo[3.1.0]hex-2-ylidenemethyl]-benzoic acid
4-[1-(5,5,8,8-Tetramethyl-5,6,7,8-tetrahydro-naphthalen-2-yl)-bicyclo[3.1.0]hex-2-ylidenemethyl]-benzoic acid,
6-[1-(3,5,5,8,8-Pentamethyl-5,6,7,8-tetrahydro-naphthalen-2-yl)-bicyclo[3.1.0]hex-2-ylidenemethyl]-nicotinic acid,
6-[1-(5,5,8,8-Tetramethyl-5,6,7,8-tetrahydro-naphthalen-2-yl)-bicyclo[3.1.0]hex-2-ylidenemethyl]-nicotinic acid,
4-[2-(3,5,5,8,8-Pentamethyl-5,6,7,8-tetrahydro-naphthalen-2-yl)-cyclopentylidenemethyl]-benzoic acid,
4-[2-(5,5,8,8-Tetramethyl-5,6,7,8,8-tetrahydro-naphthalen-2-yl)-cyclopentylidenemethyl]-benzoic acid,
4-[2-(3,5,5,8,8-Pentamethyl-5,6,7,8-tetrahydro-naphthalen-2-yl)-cyclopentylidenemethyl]-nicotinic acid,
4-[2-(5,5,8,8-Tetramethyl-5,6,7,8-tetrahydro-naphthalen-2-yl)-cyclopentylidenemethyl]-nicotinic acid,
4-[1-(3,5,5,8,8-Pentamethyl-5,6,7,8-tetrahydro-naphthalen-2-yl)-bicyclo[3.1.0]hex-2-en-2-yl]-benzoic acid,
4-[1-(5,5,8,8-Tetramethyl-5,6,7,8-tetrahydro-naphthalen-2-yl)-bicyclo[3.1.0]hex-2-en-2-yl]-benzoic acid,
6-[1-(3,5,5,8,8-Pentamethyl-5,6,7,8-tetrahydro-naphthalen-2-yl)-bicyclo[3.1.0]hex-2-en-2-yl]-nicotinic acid,
6-[1-(5,5,8,8-Tetramethyl-5,6,7,8-tetrahydro-naphthalen-2-yl)-bicyclo[3.1.0]hex-2-en-2-yl]-nicotinic acid,
3-[1-(3,5,5,8,8-Pentamethyl-5,6,7,8-tetrahydro-naphthalen-2-yl)-bicyclo[3.1.0]hex-2-en-2-yl]-acrylic acid,
3-[1-(5,5,8,8-Tetramethyl-5,6,7,8-tetrahydro-naphthalen-2-yl)-bicyclo[3.1.0]hex-2-en-2-yl]-acrylic acid,
[1-(3,5,5,8,8-Pentamethyl-5,6,7,8-tetrahydro-naphthalen-2-yl)-bicyclo[3.1.0]hex-2-en-2-yl]-propynoic acid,
[1-(5,5,8,8-Tetramethyl-5,6,7,8-tetrahydro-naphthalen-2-yl)-bicyclo[3.1.0]hex-2-en-2-yl]-propynoic acid,
[2-(3,5,5,8,8-Pentamethyl-5,6,7,8-tetrahydro-naphthalen-2-yl)-cyclopent-2-enylidene]-acetic acid,
[2-(5,5,8,8-Tetramethyl-5,6,7,8-tetrahydro-naphthalen-2-yl)-cyclopent-2-enylidene]-acetic acid,
4-[2-(3,5,5,8,8-Pentamethyl-5,6,7,8-tetrahydro-naphthalen-2-yl)-cyclopent-2-enylidenemethyl]-benzoic acid,
4-[2-(5,5,8,8-Tetramethyl-5,6,7,8-tetrahydro-naphthalen-2-yl)-cyclopent-2-enylidenemethyl]-benzoic acid,
6-[2-(3,5,5,8,8-Pentamethyl-5,6,7,8-tetrahydro-naphthalen-2-yl)-cyclopent-2-enylidenemethyl]-nicotinic acid,
6-[2-(5,5,8,8-Tetramethyl-5,6,7,8-tetrahydro-naphthalen-2-yl)-cyclopent-2-enylidenemethyl]-nicotinic acid,
4-[3-Oxo-2-(3,5,5,8,8-pentamethyl-5,6,7,8-tetrahydro-naphthalen-2-yl)-cyclopentyl]-benzoic acid,
4-[3-Oxo-2-(5,5,8,8-tetramethyl-5,6,7,8-tetrahydro-naphthalen-2-yl)-cyclopentyl]-benzoic acid,
4-[3-Methylene-2-(3,5,5,8,8-pentamethyl-5,6,7,8-tetrahydro-naphthalen-2-yl)-cyclopentyl]-benzoic acid,
4-[3-Methylene-2-(5,5,8,8-tetramethyl-5,6,7,8-tetrahydro-naphthalen-2-yl)-cyclopentyl]-benzoic acid,
4-[4-(3,5,5,8,8-Pentamethyl-5,6,7,8-tetrahydro-naphthalen-2-yl)-spiro[2.4]hept-5-yl]-benzoic acid,
4-[4-(5,5,8,8-Tetramethyl-5,6,7,8-tetrahydro-naphthalen-2-yl)-spiro[2.4]hept-5-yl]-benzoic acid,
3-{4-[2-(3,5,5,8,8-Pentamethyl-5,6,7,8-tetrahydro-naphthalen-2-yl)-cyclopent-1-enyl]-phenyl}-but-2-enoic acid,
3-{4-[2-(5,5,8,8-Tetramethyl-5,6,7,8-tetrahydro-naphthalen-2-yl)-cyclopent-1-enyl]-phenyl}-but-2-enoic acid,
3-{6-[2-(3,5,5,8,8-Pentamethyl-5,6,7,8-tetrahydro-naphthalen-2-yl)-cyclopent-1-enyl]-pyridin-3-yl}-but-2-enoic acid,
3-{6-[2-(5,5,8,8-Tetramethyl-5,6,7,8-tetrahydro-naphthalen-2-yl)-cyclopent-1-enyl]-pyridin-3-yl}-but-2-enoic acid,
4-[2-(3,5,5,8,8-Pentamethyl-5,6,7,8-tetrahydro-naphthalen-2-yl)-cyclopent-2-enyl]-benzoic acid,
4-[2-(5,5,8,8-Tetramethyl-5,6,7,8-tetrahydro-naphthalen-2-yl)-cyclopent-2-enyl]-benzoic acid,
6-[2-(3,5,5,8,8-Pentamethyl-5,6,7,8-tetrahydro-naphthalen-2-yl)-cyclopent-2-enyl]-nicotinic acid,
6-[2-(5,5,8,8-Tetramethyl-5,6,7,8-tetrahydro-naphthalen-2-yl)-cyclopent-2-enyl]-nicotinic acid,
[2-(3,5,5,8,8-Pentamethyl-5,6,7,8-tetrahydro-naphthalen-2-yl)-cyclopent-2-enyl]-propynoic acid,
[2-(5,5,8,8-Tetramethyl-5,6,7,8-tetrahydro-naphthalen-2-yl)-cyclopent-2-enyl]-propynoic acid,
3-[2-(3,5,5,8,8-Pentamethyl-5,6,7,8-tetrahydro-naphthalen-2-yl)-cyclopent-2-enyl]-acrylic acid,
3-[2-(5,5,8,8-Tetramethyl-5,6,7,8-tetrahydro-naphthalen-2-yl)-cyclopent-2-enyl]-acrylic acid,
4-[1-Methoxy-2-(3,5,5,8,8-pentamethyl-5,6,7,8-tetrahydro-naphthalen-2-yl)-cyclopentyl]-benzoic acid,
4-[1-Methoxy-2-(5,5,8,8-tetramethyl-5,6,7,8-tetrahydro-naphthalen-2-yl)-cyclopentyl]-benzoic acid,
6-[1-Methoxy-2-(3,5,5,8,8-pentamethyl-5,6,7,8-tetrahydro-naphthalen-2-yl)-cyclopentyl]-nicotinic acid,
6-[1-Methoxy-2-(5,5,8,8-tetramethyl-5,6,7,8-tetrahydro-naphthalen-2-yl)-cyclopentyl]-nicotinic acid,
[1-Methoxy-2-(3,5,5,8,8-pentamethyl-5,6,7,8-tetrahydro-naphthalen-2-yl)-cyclopentyl]-propynoic acid,
[1-Methoxy-2-(5,5,8,8-tetramethyl-5,6,7,8-tetrahydro-naphthalen-2-yl)-cyclopentyl]-propynoic acid,
3-[1-Methoxy-2-(3,5,5,8,8-pentamethyl-5,6,7,8-tetrahydro-naphthalen-2-yl)-cyclopentyl]-acrylic acid,
3-[1-Methoxy-2-(5,5,8,8-tetramethyl-5,6,7,8-tetrahydro-naphthalen-2-yl)-cyclopentyl]-acrylic acid,
4-[2-Methoxy-1-(3,5,5,8,8-pentamethyl-5,6,7,8-tetrahydro-naphthalen-2-yl)-bicyclo[3.1.0]hex-2-yl]-benzoic acid,
4-[2-Methoxy-1-(5,5,8,8-tetramethyl-5,6,7,8-tetrahydro-naphthalen-2-yl)-bicyclo[3.1.0]hex-2-yl]-benzoic acid,
6-[2-Methoxy-1-(5,5,8,8-tetramethyl-5,6,7,8-tetrahydro-naphthalen-2-yl)-bicyclo[3.1.0]hex-2-yl]-nicotinic acid,
6-[2-Methoxy-1-(3,5,5,8,8-pentamethyl-5,6,7,8-tetrahydro-naphthalen-2-yl)-bicyclo[3.1.0]hex-2-yl]-nicotinic acid,
[2-Methoxy-1-(3,5,5,8,8-pentamethyl-5,6,7,8-tetrahydro-naphthalen-2-yl)-bicyclo[3.1.0]hex-2-yl]-propynoic acid,
[2-Methoxy-1-(5,5,8,8-tetramethyl-5,6,7,8-tetrahydro-naphthalen-2-yl)-bicyclo[3.1.0]hex-2-yl]-propynoic acid,
3-[2-Methoxy-1-(3,5,5,8,8-pentamethyl-5,6,7,8-tetrahydro-naphthalen-2-yl)-bicyclo[3.1.0]hex-2-yl]-acrylic acid,
3-[2-Methoxy-1-(5,5,8,8-tetramethyl-5,6,7,8-tetrahydro-naphthalen-2-yl)-bicyclo[3.1.0]hex-2-yl]-acrylic acid,
4-[1-Methoxy-2-(3,5,5,8,8-pentamethyl-5,6,7,8-tetrahydro-naphthalen-2-yl)-cyclopent-2-enyl]-benzoic acid,
4-[1-Methoxy-2-(5,5,8,8-tetramethyl-5,6,7,8-tetrahydro-naphthalen-2-yl)-cyclopent-2-enyl]-benzoic acid,
6-[1-Methoxy-2-(3,5,5,8,8-pentamethyl-5,6,7,8-tetrahydro-naphthalen-2-yl)-cyclopent-2-enyl]-nicotinic acid,
6-[1-Methoxy-2-(5,5,8,8-tetramethyl-5,6,7,8-tetrahydro-naphthalen-2-yl)-cyclopent-2-enyl]-nicotinic acid,
[1-Methoxy-2-(3,5,5,8,8-pentamethyl-5,6,7,8-tetrahydro-naphthalen-2-yl)-cyclopent-2-enyl]-propynoic acid,
[1-Methoxy-2-(5,5,8,8-tetramethyl-5,6,7,8-tetrahydro-naphthalen-2-yl)-cyclopent-2-enyl]-propynoic acid,
3-[1-Methoxy-2-(3,5,5,8,8-pentamethyl-5,6,7,8-tetrahydro-naphthalen-2-yl)-cyclopent-2-enyl]-acrylic acid, and
3-[1-Methoxy-2-(5,5,8,8-tetramethyl-5,6,7,8-tetrahydro-naphthalen-2-yl)-cyclopent-2-enyl]-acrylic acid,
or a pharmaceutically acceptable salt thereof.

6. A compound according to claim 1 which acts as a Retinoid X Receptor (RXR) agonist.

7. A pharmaceutical composition comprising, as an active ingredient, an effective amount of a compound of claim 1 or a pharmaceutically acceptable salt thereof, together with a pharmaceutically acceptable carrier or diluent.

8. The composition according to claim 7 in unit dosage form, comprising from about 0.05 to about 100 mg of the compound or pharmaceutically acceptable salt thereof.

9. The pharmaceutical composition according to claim 7 for oral, nasal, transdermal, pulmonal, or parenteral administration.

10. A method for the treatment of non-insulin dependent diabetes mellitus, the method comprising administering to a subject in need thereof an effective amount of a compound of claim 1 or the composition of claim 7.

11. The method according to claim 10, wherein the effective amount of the compound is in the range of from about 0.05 to about 100 mg per day.
